(19) United States
(12) Patent Application Publication
(10) Pub. No.: US 2008/0009878 A1
(43) Pub. Date: Jan. 10, 2008

(54) Fissure Repair

(76) Inventors: Alan McLeod, Somerset (GB); Christopher Reah, Taunton (GB)

Correspondence Address: JONATHAN SPANGLER;NU VASIVE, INC., 4545 TOWNE CENTRE COURT, SAN DIEGO, CA, 92121, US

(21) Appl. No.: 10/594,158

(22) PCT Filed: Mar. 24, 2005
(86) PCT No.: PCT/GB05/01157

§ 371 Date: Aug. 2, 2007

(30) Foreign Application Priority Data
Mar. 26, 2004 (GB) 0406835.9

(51) Int. Cl. A61B 17/56

(52) U.S. Cl. 606/86

(57) ABSTRACT

A fissure repair (60) device for a spinal disc is provided, the device including a first portion (61), a second portion (62a, 62b) and a variable link (63a, 63b) between the first portion and second portion, in which the first and second portions are portions of a common element, one of the first or second portions being formed by one or both end portions of the element, the first portion being linked to the second portion by one or more link portions (63a, 63b), the one or more link portions being portions of the common element.

This invention concerns improvements in and relating to fissure repair, for instance wound/incision closure, particularly, but not exclusively in relation to the closure of wounds/incisions in intervertebral discs.

A number of surgical techniques are known with a view to addressing problems with intervertebral discs. Such techniques include the insertion of devices to attempt to restore the natural function of a disc or to prevent further deterioration in performance. Techniques are also known which attempt to repair damage to intervertebral discs.

In the case of the insertion of an artificial nucleus for a disc, a series of incisions are usually made in the annulus of the disc, a part or all of the nucleus is removed and the replacement device is then inserted. The incisions in the annulus are then closed and sutured. The sutures are applied direct to the annulus itself. The suture is passed through the annulus on one side of the incision, bridges the incision and is then passed through the annulus on the other side. Repeats of this process are used to try and close the incision.

This approach relies upon the annulus itself to provide the anchorage for the sutures. The loads involved, coupled potentially with impaired structural properties for the annulus make this suturing process less than optimal. Additionally, the incision is fully exposed to pressure from the nucleus and/or device and this can cause the incision to open up. The problems are particularly significant with the annulus and other components of an intervertebral disc as from around 20 years onward they become avascular and hence heal poorly.

The present invention has amongst its aims potentially to provide a repair device for an intervertebral disc, a method of repairing an intervertebral disc, a kit including a repair device for an intervertebral disc. The present invention has amongst its aims potentially to provide a stronger anchorage for suturing to close a wound/incision. The present invention has amongst its aims potentially to provide protection for a wound/incision against internal pressure. The present invention has amongst its aims potentially to provide improved recovery of the intervertebral disc or parts thereof. The present invention has amongst its aims potentially to provide an improved device and/or method for returning or maintaining intervertebral disc function.

According to a first aspect of the invention we provide fissure repair device, the device including a first portion, a second portion and a variable link between the first portion and second portion.

Preferably the device is an assembled device.

The fissure may be caused by an incision and/or a wound and/or an injury and/or degeneration and/or disease.

The repair may be closure. The repair may refer to bringing the sides of the fissure together and/or bringing the sides of the fissure into proximity with one another and/or bringing, and preferably maintaining, the sides of the fissure in position relative to one another.

The first portion may be used within a vertebral disc, particularly inside at least part of the annulus thereof.

The first portion may be fabric. The first portion may be woven and/or embroidered and/or knitted and/or braided and/or combinations thereof. The first portion may be of textile. The first portion may be provided with embroidery. The first portion may be provided with one or more areas of reinforcement. The one or more areas of reinforcement may be provided by further weaving and/or embroidery.

The first portion may be provided with one or more holes. The first portion may be provided with a first set of holes and preferably also a second set of holes. A first set of holes may be provided towards one end of the first portion. A second set of holes may be provided towards the other end of the first portion. The holes in a set may be provided singly in line. The line may extend along the height of the first portion. Preferably a set of holes includes four holes. Preferably, in the case of the first portion, the holes are constructed by the weaving process.

Alternatively, the holes may be provided by the gaps between the fibres forming the woven fabric. One or more areas of reinforcement may be provided around one or more holes in the first portion.

Particularly in the case of the first portion, the first portion may be evenly provided all over. Alternatively, one or more areas of reinforcement may be provided along a central part of the first portion. The central part may extend for the entire length of the first portion. The central part may extend for over 60% of the length of the first portion. The central part may extend for less than 90% of the length of the first portion. The central part may extend for only part of the height of the first portion, for instance between 10% and 80% thereof. Preferably the central part extends evenly in terms of length and/or height about the centre of the first portion.

The first portion may be of polyester. The first portion may be made of one or more materials and/or incorporate one or more materials and/or be coated with one or more materials which promote tissue in growth and/or the supply of blood. The first portion may include glass fibre and/or alginate and/or polyester and/or polypropylene and/or polyethylene and/or glass and/or polyaramide and/or metal and/or copolymers and/or polylactic acid and/or polyglycolic acid and/or biodegradable materials and/or silk and/or cellulose and/or polycaprolactone materials.

One or more or all of the materials defining the first portion may be bio-absorbable. The time period required for bio-absorption may vary between different materials used to define the first portion.

The first portion is preferably flexible. Preferably the first portion can adopt the profile of a surface it is pulled and/or pushed against. The first portion may be pushed against an annulus surface due to the pressure exerted upon it by a device within the annulus and/or the nucleus within the annulus. The first portion may be pulled against an annulus surface due to the variable link.

The first portion preferably has a length greater than its height and/or thickness. Within this application, preferably, height is measured along the spine, thickness is measured into the spine and length is measured perpendicular to both of those. The length may be between 1.5 and 5 times the height. Preferably the length is at least 5 times the width of the fissure after closure, more preferably at least 10 times.

The first portion may be generally rectilinear, potentially with rounded corners. The first portion may have linear edges, preferably on top and bottom and/or both side edges. One or both pairs of edges may be parallel to one the other in the pair.

The first portion may be provided with one or more expanding components. The one or more expanding components may have a first contracted state and a second expanded state. Preferably the component undergoes a transition from first to second state during deployment, and ideally after passage through the fissure. The one or more components may expand due to the removal of a restraining force. The one or more components may expand due to a change in state, such as for a memory metal. The one or more components may be provided in a configuration capable of expansion, such as a star, spiral or the like. Preferably in the first state the first portion can be readily passed through the fissure. Preferably in the second state the first portion is larger than the fissure. The expandable component may be provided in a pocket in the first portion or on the first portion. Preferably the pocket is defined between the second part and the first and third parts of the first portion.

The first portion may be provided with a through aperture, particularly in the middle thereof. The aperture may be used to provide a passage between the outside of the annulus and the inside, via the fissure and aperture. The aperture may have an open state and a closed state. Preferably in the closed state, a component covers the aperture. The component preferably covers the inside of the aperture. The component may be a flap, preferably provided on the first portion and ideally provided as an integral part thereof. In the open state, the component may be positioned away from the aperture, for instance above it. The aperture may lead to the inside of the annulus and/or to the nucleus and/or to a device of the type detailed in UK Patent Application no 0406851.6 filed 26 Mar. 2004 and/or UK Patent Application No 0407717.8 filed 5 Apr. 2004.

The aperture may lead to an enclosure, for instance within the nucleus. The enclosure may be defined by a bag or sack. The enclosure may be impermeable to hydrogel. The hydrogel may be introduced in an non-set state and allowed to set in-situ. The enclosure may be impermeable to a flowable core forming material. The flowable core forming material may set in-situ to form a core. Preferably the device maintains the position of the enclosure relative to the inside of the disc.

The enclosure may be provided with a core, for instance a pre-formed core. The core may be one or more elastomeric components. The core may be provided in the enclosure before insertion of the device into the disc and/or after insertion of the device into the disc.

The second portion may be used outside a vertebral disc, particularly outside the annulus thereof.

The second portion may be fabric. The second portion may be woven and/or embroidered. The second portion may be of textile. The second portion may be provided with embroidery. The second portion may be provided with one or more areas of reinforcement. The one or more areas of reinforcement may be provided by further weaving and/or embroidery.

The second portion may be provided with one or more holes. Preferably, in the case of the second portion, the holes are provided by the gaps between the fibres forming the woven fabric. Alternatively, the holes are constructed by the weaving process. The second portion may be provided with a first set of holes and preferably also a second set of holes. A first set of holes may be provided towards one end of the second portion. A second set of holes may be provided towards the other end of the second portion. The holes in a set may be provided singly in line. The line may extend along the height of the second portion. Preferably a set of holes includes four holes. One or more areas of reinforcement maybe provided around one or more holes in the first portion.

Particularly in the case of the second portion, one or more areas of reinforcement may be provided along a central part of the first portion. The central part may extend for the entire length of the first portion. The central part may extend for over 60% of the length of the first portion. The central part may extend for less than 90% of the length of the first portion. The central part may extend for only part of the height of the first portion, for instance between 10% and 80% thereof. Preferably the central part extends evenly in terms of length and/or height about the centre of the first portion. Alternatively, the second portion may be evenly provided all over.

The second portion may be of polyester. The second portion may be made of one or more materials and/or incorporate one or more materials and/or be coated with one or more materials which promote tissue in growth and/or the supply of blood. The second portion may include glass fibre and/or alginate and/or carbon fibre materials.

One or more or all of the materials defining the second portion may be bio-absorbable. The time period required for bio-absorption may vary between different materials used to define the second portion.

The second portion is preferably flexible. Preferably the second portion can adopt the profile of a surface it is pulled and/or pushed against. The second portion may be pulled against an outside surface of the annulus due to the variable link.

The second portion preferably has a length greater than its height and/or thickness. The length may be between 1.5 and 5 times the height. Preferably the length is at least 5 times the width of the fissure after closure, more preferably at least 10 times.

The second portion maybe generallyrectilinear, potentially with rounded corners. The second portion may have linear edges, preferably on top and bottom and/or both side edges. One or both pairs of edges may be parallel to one the other in the pair.

The first and second portions may have the same length and/or height and/or thickness as each other. The first and second portions may be provided of the same material. The first and second portions maybe provided with reinforcement and/or holes in the same positions and/or over the same areas.

The first portion and second portion may be separate from one another.

The first portion and second portion may be integral portions of a single element.

The variable link may be a thread, suture or stitch. Needles may be attached to or integrally provided on the variable link, at one or more ends. The needles may be incorporated in the first portion, for instance between the second part of the first portion and the first and third parts of the first portion. The needles may partially protrude through the first portion or a part thereof. The slack in the variable link may be provided between parts of the first portion, preferably between the second part of the first portion and the first and third parts of the first portion. Preferably at least two variable links are provided. A variable link may include one or more parts thereof which link the first portion to the second portion. Preferably two such parts are provided for each link. Preferably the two parts are provided by different but integral parts of the link. Preferably one variable link connects a first end of the first portion to a first end of the second portion. Preferably the first end of the first portion and first end of the second portion are those ends which are closest to one another in the assembled form. Preferably one variable link connects a second end of the first portion to a second end of the second portion. Preferably the second end of the first portion and second end of the second portion are those ends which are closest to one another in the assembled form.

Preferably the variable link can be used to vary the distance between the first portion and the second portion. Preferably the variable link can be used to vary the tension between the first portion and the second portion. Preferably the variable link is used to pull the first portion against the inside of the annulus or a part thereof and/or to pull the second portion against the outside of the annulus.

Preferably the variable link can be threaded through the first portion, for instance through one or more holes. Preferably at least the part of the first portion that the variable link passes through is reinforced. The variable link may pass from the front of the first portion, through the first portion, round the back thereof and through the first portion to the front once more. The variable link may further pass in front of the first portion, through the first portion, round the back thereof and through the first portion to the front thereof. Preferably the variable link is free to move relative to the first portion.

Preferably the variable link can be threaded through the second portion, for instance through one or more gaps in the second portion. Preferably the variable link is threaded through a part of the second portion to one side of a reinforced area. Preferably it then passes over the reinforced area before passing through a part of the second portion on the other side of the reinforced area. The variable link may be tied into a knot on the front surface of the second portion. Preferably the knot is tied against a part of the second portion that is reinforced. Preferably the variable link is free to move relative to the second portion prior to tying. Preferably the variable link is fixed relative to the second portion after tying.

Preferably an annulus receiving space is defined between the first portion and the second portion. Preferably a receiving space for the annulus to one side of the fissure is provided between the first end of the first portion and the first end of the second portion. Preferably a receiving space for the annulus to the other side of the fissure is provided between the second end of the first portion and the second end of the second portion.

Preferably the holes in the first and/or second portions oppose an annulus receiving space. Preferably the holes in the first end and/or second end of the first portion and/or the first end and/or second end of the second portions oppose an annulus receiving space.

Preferably the variable link passes through an annulus receiving space, particularly in use. Preferably the variable link at the first end of the first portion passes through an annulus receiving space on its passage to the first end of the second portion. Preferably the variable link at the second end of the first portion passes through an annulus receiving space on its passage to the second end of the second portion.

Preferably the first portion extends to either side of the fissure. Preferably the extent is greater than 5 times the width of the fissure. Preferably the first portion extends for the full height of the fissure. Preferably the second portion extends to either side of the fissure. Preferably the extent is greater than 5 times the width of the fissure. Preferably the second portion extends for the full height of the fissure.

Preferably the first portion provides a continuous portion across the inside of the fissure. The second portion may provide a continuous portion across the outside of the fissure.

The first and/or second portions may be fastened to the annulus by one or more fixings. The fixings may be staples or barbs or the like, preferably incorporated in the first and/or second portion. The fixings may protrude through the first portion, particularly through the first and third parts thereof.

Any of the features, options or possibilities set out above in relation to the first aspect of the invention or elsewhere in this document may be used in the other aspects of the invention.

According to a second aspect of the invention we provide an assembled fissure repair device, the device including a first portion, a second portion and a variable link between the first portion and second portion, in which the first and second portions are portions of a common element, one of the first or second portions being formed by one or both end portions of the element, the first portion being linked to the second portion by one or more link portions, the one or more link portions being portions of the common element.

Preferably the other of the first or second portion is formed by an intermediate portion of the element. Ideally the first portion is formed by the intermediate portion of the element. Ideally the second portion is formed by the two end portions of the element. The element may be in the form of a first second portion forming part, first link portion, first portion, second link portion and second second portion forming part, ideally with this being the sequence from one end to the other of the element.

The first portion may include a first part, second part and third part. Preferably the parts are all integral to the first portion. Preferably the boundaries between first and second parts and/or between second and third parts are defined by a fold.

The first portion may be provided with one or more holes, particularly in the second part thereof. The one or more holes in the second part thereof may be provided in the form of a first set of holes and a second set of holes. A first set of holes may be provided towards one end of the second part of the first portion. The second set of holes may be provided towards the other end of the second part of the first portion. The holes in a set may be provided singly in line. The line may extend along the height of the first portion. Preferably a set of holes includes four holes. Preferably, in the case of the first portion, the holes are constructed by the weaving process.

The first portion, particularly the first and third parts thereof, may be provided with one or more further sets of holes. Preferably the first part is provided with one set of holes, ideally with the set including two holes. Preferably the third part is provided with one set of holes, ideally with the set including two holes. Preferably when the first part and/or third part are folded against the second part, the holes in the first and third parts align with holes in the second part.

The second portion may be provided with one or more areas of reinforcement. Preferably an area of reinforcement is provided on each of the end portions of the element. The reinforcement may be provided towards the middle of the end portions, relative to their length and/or height. The one or more areas of reinforcement may be provided by further weaving and/or embroidery.

Preferably, in the case of the second portion, the holes are provided by the gaps between the fibres forming the woven fabric.

The link portion(s) may be made of one or more materials and/or incorporate one or more materials and/or be coated with one or more materials which promote tissue in growth and/or the supply of blood. The link portion(s) may include glass fibre and/or alginate and/or carbon fibre materials.

One or more or all of the materials defining the link portion(s) may be bio-absorbable. The time period required for bio-absorption may vary between different materials used to define the link portion(s).

A fold maybe provided between the first second portion forming part and the first link portion and/or between the second link portion and the second second portion forming part. The folds maybe of 90°+/−5°. A fold maybe provided between the first link portion and the first portion and/or between the first portion and the second link portion. The folds may be of 90°+/−5°. A fold may be provided between a first part of the first portion and a second part of the first portion and/or between a third part of the first portion and a second part of the first portion. The folds may be of 360°+/−5°.

When folded, preferably the first second portion forming part and second second portion forming part lie substantially on the same plane and/or represent a continuous arc. One or preferably both of the second portion forming parts may contact the outside of the annulus and/or oppose the first portion.

When folded, preferably the first link portion contacts the second link portion. The first and/or second link portions may be substantially parallel to one another. The first and/or second link portions may be at 90°+/−5° to the first portion and/or second portion. The first and/or second link portions may contact the sides of the fissure.

When folded, preferably the first and third parts of the first portion contact the inside wall of the annulus and/or oppose the second portion. Preferably the second part of the first portion is folded against the first and third parts and/or is separated from the second portion by the first and third parts of the first portion. The first and/or third parts of the first portion may be parallel and/or concentric with the second part of the first portion.

Preferably the first portion and second portion define the verticals of an H shape, particularly when considered in plan view in an intervertebral disc space. Preferably the link portions define the cross bar of an H shape.

The first portion and/or second portion and/or link portion may be of double thickness of material, for instance due to folding.

Preferably at least two variable links are provided. Preferably one is provided between one end of the first portion, preferably the second part thereof, and the second portion. Preferably the other is provided between the other end of the first portion, preferably the second part thereof, and the second portion. Preferably the variable links pass through the first part of the first portion and the third part of the second. Preferably each of the variable links is in the form of two parts provided by different but integral parts of the link.

Preferably a variable link can be used to vary the distance between one end of the first portion and the same end of the second portion. Preferably the variable link can be used to vary the tension between one end of the first portion and the same end of the second portion. Preferably the variable link is used to pull the first portion against the inside of the annulus or a part thereof and/or to pull the second portion against the outside of the annulus. Preferably the variable link causes the linking portion or portions to pull out through the fissure to the outside of the annulus.

Preferably a receiving space for the annulus to one side of the fissure is provided between a first end of the first portion and a first end of the second portion. For instance, the receiving space may be provided between one end of the element and the first part of the first portion. Preferably a receiving space for the annulus to the other side of the fissure is provided between a second end of the first portion and a second end of the second portion. For instance, the receiving space may be provided between the other one end of the element and the third part of the first portion.

Preferably the link portion or portions pass through the fissure, ideally from inside the annulus to the outside thereof. Preferably the link portion or portions keep the sides of the fissure apart.

Any of the features, options or possibilities set out above in relation to the second aspect of the invention or elsewhere in this document may be used in the other aspects of the invention.

According to a third aspect of the invention we provide fissure repair device, the device including a first portion, a second portion and a variable link between the first portion and second portion, in which the first and second portions are portions of a common element, the first portion being linked to the second portion by one or more link portions, the one or more link portions being portions of the common element, the second portion being formed by both end portions of the element.

Preferably the first portion is formed by an intermediate portion of the element. The element may be in the form of a first second portion forming part, first link portion, first portion, second link portion and second second portion forming part, ideally with this being the sequence from one end to the other of the element. One of the second portion forming parts and/or the link portion connected to it, may be provided with a reduced height part and/or neck part. The other of the second portion forming parts and/or the link portion connected to it, may be provided with an aperture. The neck part and/or rim of the aperture may be reinforced. Preferably the height of the reduced height portion and/or neck part is less than the height of the aperture.

The second portion forming part provided with the reduced height part and/or neck part may be provided with one or more areas of reinforcement closer to the end of that part than the reduced height part and/or neck part. Preferably an area of reinforcement is provided on each of the end portions of the element. The reinforcement may be provided towards the middle of the end portions, relative to their length and/or height. The one or more areas of reinforcement may be provided by further weaving and/or embroidery.

Preferably, in the case of the second portion, the holes are provided by the gaps between the fibres forming the woven fabric.

A fold may be provided between the first second portion forming part and the first link portion, preferably passing through the reduced height part and/or neck part. Additionally or alternatively, a fold may be provided between the second link portion and the second second portion forming part, preferably passing through the aperture. The folds may be of 90°+/−5°.

Preferably the second portion forming part provided with the reduced height part and/or neck part is passed through the hole in the other second portion forming part. Preferably the second portion forming part provided with the reduced height part and/or neck part is passed through the hole in the other second portion forming part, to such an extent that the reduced height part and/or neck part is in the hole. Preferably the first second portion forming part is interdigitated with the second second portion forming part.

When folded, preferably the first second portion forming part and second second portion forming part lie substantially on the same plane and/or represent a continuous arc, but on the opposite side of the fissure to the link portion to which they are connected. One or preferably both of the second portion forming parts may contact the outside of the annulus and/or oppose the first portion, but on the opposite side of the fissure to the link portion to which they are connected.

Preferably a variable link can be used to reduce the distance between one side of the fissure and the other side of the fissure.

Any of the features, options or possibilities set out above in relation to the third aspect of the invention or elsewhere in this document may be used in the other aspects of the invention.

According to a fourth aspect of the invention we provide a kit for forming a fissure repair device, the kit including a first portion, a second portion and a variable link.

The variable link may be provided connected to the first portion. The variable link may be connected to the first portion by being integral thereto. The variable link may be one or more continuations of the material forming the first portion, for instance fibres thereof. The variable link may be connected to the first portion by threading the variable link through the first portion, for instance through one or more holes.

The variable link may be provided separate from the second portion.

The kit may include one or more needles. The needles may be separate from, but connectable to the variable link. The needles may be integrally provided with the variable link.

The kit may include a first portion and second portion which are provided by a common element.

Any of the features, options or possibilities set out above in relation to the fourth aspect of the invention or elsewhere in this document may be used in the other aspects of the invention.

According to a fifth aspect of the invention we provide a method of repairing a fissure in a material, the method including the steps of:
providing a fissure repair device, the device including a first portion, a second portion and a variable link;
deploying the first portion of the device inside the fissure;
deploying the second portion of the device outside the fissure;
connecting the first portion to the second portion at one or more locations using the variable link, the variable link passing through the material.

The fissure may be caused by an incision and/or a wound and/or an injury and/or degeneration and/or disease. The material is preferably the annulus of an intervertebral disc.

The repair may involve closure and/or may refer to bringing the sides of the fissure together and/or bringing the sides of the fissure into proximity with one another and/or bringing, and preferably maintaining, the sides of the fissure in position relative to one another.

The device may be provided with the variable link connected to the first portion. The method may include connecting the variable link to the first portion.

Preferably the first portion is deployed without the variable link being connected to the second portion.

Preferably the first portion is deployed through the fissure to the inside. Preferably the first portion is deployed inside the annulus. The first portion may be deployed using an applicator, for instance such as the type of applicator provided in the sixth aspect of the invention. The width of the fissure may be increased temporarily to aid deployment of the first portion.

Preferably once through the fissure, the first portion is deployed to either side of the fissure. Preferably in the deployed position the first portion extends away from the fissure on either side. Preferably the first portion provides a continuous cover for the fissure. Preferably the first portion obscures the fissure when viewed from inside. Preferably the first portion provides a barrier between the inside of the annulus and the fissure. The first portion may be pushed against the inside wall of the annulus, for instance by the nucleus and/or a device provided inside the annulus.

Preferably the variable link is passed through the material, ideally the annulus. Preferably one part of a variable link passes through the material to one side of the fissure, with most preferably another one part of a variable link passing through the material to the other side of the fissure. Preferably different parts of a variable link are provided for this purpose, most preferably different variable links are used. Preferably two parts of a variable link are passed through the material to one side of the fissure and two parts of another variable link are passed through the material to the other side of the fissure. Preferably the variable link is passed through the material from inside to outside. A separate needle connected to the variable link or an integral needle may be used to assist in the passage of the variable link through the material.

Preferably the variable link is passed through the second portion after passing through the material. Preferably two such variable links are passed through the second portion and ideally two parts of two variable links are so passed. Preferably the variable fink passes through the second portion to one side of a reinforcement area. Preferably two parts of variable links are passed through the second material, one to one side of the reinforcement, the other to the other. Preferably the second portion is positioned against the outside of the annulus, ideally after passing the variable link(s) through it.

Preferably a part of a variable link is tied to another part of a variable link, ideally by knotting. The part and the another part may be parts of the same variable link. The part from one side of an area of reinforcement may be tied to another part from the other side of the area of reinforcement. Preferably a plurality of knots are provided.

The length of the variable link lying between the first portion and the second portion and/or the separation of the first and second portions may be reduced before tying the variable link. Preferably the variable link is tensioned before tying the variable link.

The device may be provided with the first portion and second portion provided by a common element.

The first portion maybe formed by folding a first part of the first portion towards a second part of the first portion and/or by folding a third part of the first portion towards a second part of the first portion. Preferably a variable link connected to the second part of the first portion is passed through the first part of the first portion and/or a variable link connected to the second part of the first portion is passed through the third part of the first portion.

Preferably the first portion is connected to the first second portion forming part and /or to the second second portion forming part by a link portion. Preferably the link portions are deployed within the fissure. Preferably the link portions extend from inside the fissure to outside the fissure.

The length of the variable link may be changed to reduce the separation of the sides of the fissure before tying the variable link.

Preferably the second portion forming part provided with a reduced height part and/or neck part is passed through a hole in the other second portion forming part. Preferably the second portion forming part provided with the reduced height part and/or neck part is passed through the hole in the other second portion forming part, to such an extent that the reduced height part and/or neck part is in the hole. Preferably the first second portion forming part is interdigitated with the second second portion forming part. Preferably these steps are performed before the variable link is connected to the second portion.

Any of the features, options or possibilities set out above in relation to the fifth aspect of the invention or elsewhere in this document may be used in the other aspects of the invention.

According to a sixth aspect of the invention we provide an applicator for a fissure repair device, the applicator including a retaining location, the retaining location holding a fissure repair device in use, an actuator for moving the fissure repair device from the retaining location.

The actuator may be a plunger or other reciprocating element. The actuator may carry one or more wires or strips extending therefrom. One or more parts of the wires or strips may engage the fissure repair device. The wires or strips may extend from the applicator in use into the disc. The wires or strips may serve to push one or more parts of the fissure repair device to the desired position within the disc. The wires or strips may be integral with the applicator and/or with the fissure repair device. Preferably the wires or strips are retractable.

The retaining location may be in the form of a chamber, preferably with a mouth adapted to be inserted into an incision in a disc.

The wires or strips may be of memory metal or other highly flexible material.

Any of the features, options or possibilities set out above in relation to the sixth aspect of the invention or elsewhere in this document may be used in the other aspects of the invention.

According to a seventh aspect of the invention we provided a fissure repair device, the device including a first portion, the first portion, on one side, being provided with one or more barbs.

The device may be used inside an intervertebral disc, for instance on the inside of an annulus. Preferably the first portion spans the fissure, in use.

Preferably one or more barbs are provided towards one end of the first portion. Preferably these barbs are inclined towards the centre of the first portion. Preferably one or more barbs are provided towards the other end of the first portion. Preferably these barbs are also inclined towards the centre of the first portion.

The device may include a second portion provided outside an intervertebral disc and/or in opposition to the first portion. Preferably the second portion spans the fissure in use.

The second portion may be attached to the annulus by one or more sutures, and preferably at least one suture on either side of the fissure.

Any of the features, options or possibilities set out above in relation to the seventh aspect of the invention or elsewhere in this document may be used in the other aspects of the invention.

According to a eighth aspect of the invention we provide a method of repairing a fissure in a material, the method including the steps of:
providing a fissure repair device, the device including a first portion, the first portion, on one side, being provided with one or more barbs;
deploying the first portion inside the fissure;
causing the barbs of the first portion to engage the material on either side of the fissure.

Preferably the material is the annulus of an intervertebral disc. Preferably the first portion spans the fissure and/or covers the fissure and/or obscures the fissure when viewed from the inside.

Preferably movement of the first portion towards the fissure and/or movement of the material away from the fissure causes one or more of the barbs to engage the material. Preferably the barbs to one side of the fissure resist movement of the sides of the fissure in one direction and the barbs to the other side of the fissure resist the movement of the sides of the fissure in the other direction.

The method may include providing a device including a second portion. The second portion may be deployed outside an intervertebral disc and/or in opposition to the first portion. Preferably the second portion spans the fissure and/or covers the fissure and/or obscures the fissure when viewed from outside.

The second portion may be attached to the annulus by one or more sutures, and preferably at least one suture on either side of the fissure. Any of the features, options or possibilities set out above in relation to the eighth aspect of the invention or elsewhere in this document may be used in the other aspects of the invention.

Various embodiments of the invention will now be described, by way of example only, and with reference to the accompanying drawings in which:

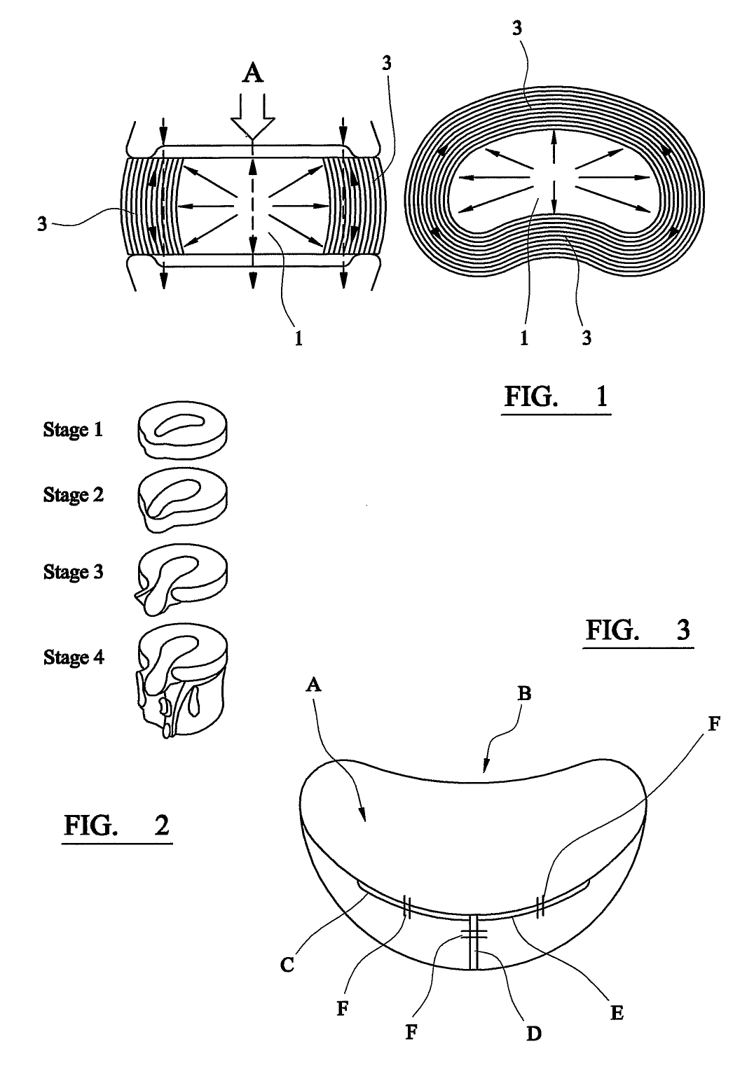

FIG. 1 illustrates the function of the annulus of an intervertebral disc;

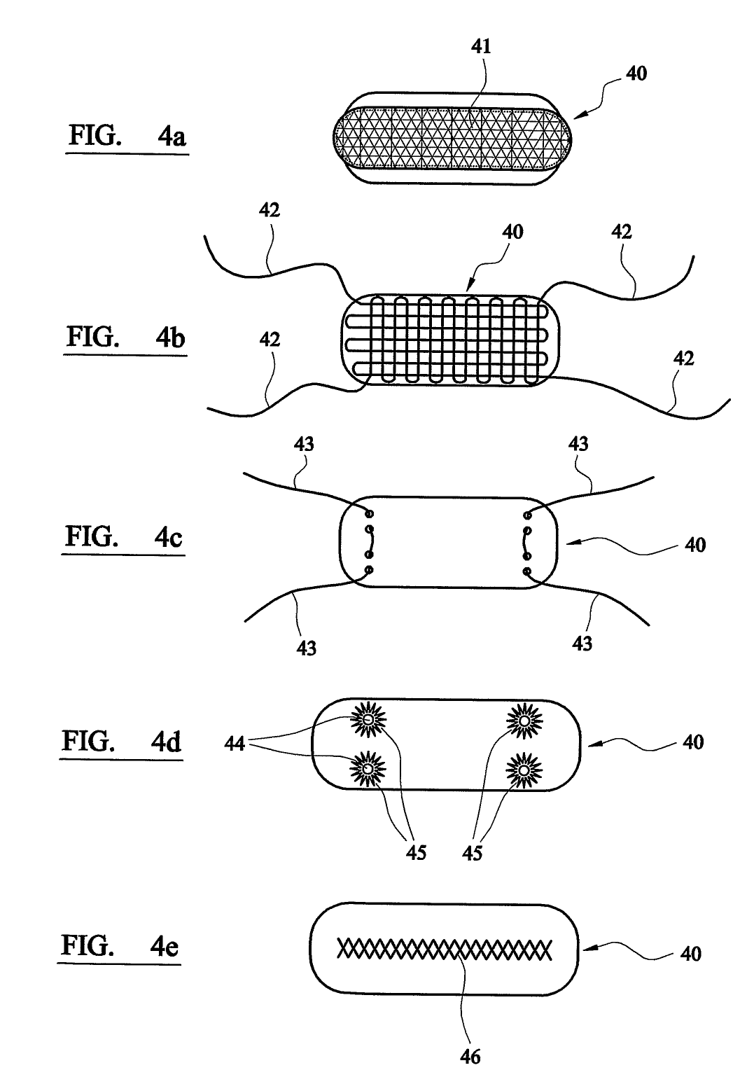

FIG. 2 illustrates the stages of disc herniation;

FIG. 3 illustrates the incision sites and optional sutures used in a prior art technique for providing an artificial disc replacement;

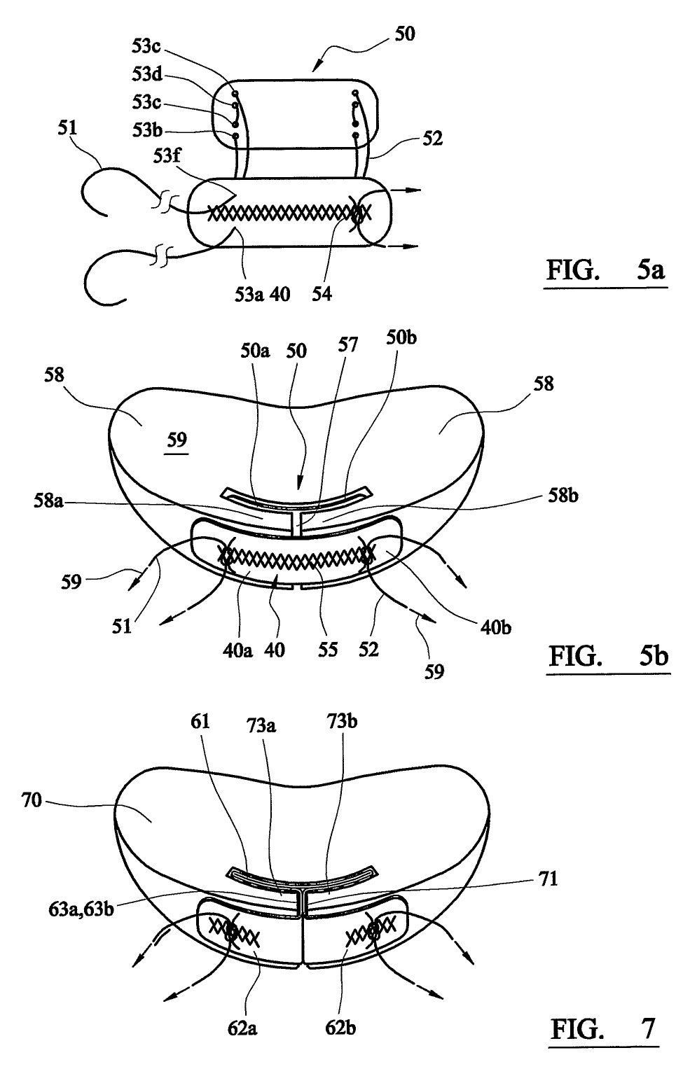

FIG. 4a to 4f illustrate one part of each of five different annular repair devices according to embodiments of the present invention;

FIG. 5a illustrates the assembly of an annular repair device according to the embodiment of FIG. 4e;

FIG. 5b illustrates the use of an assembly according to the embodiment of FIG. 4c to repair an annulus;

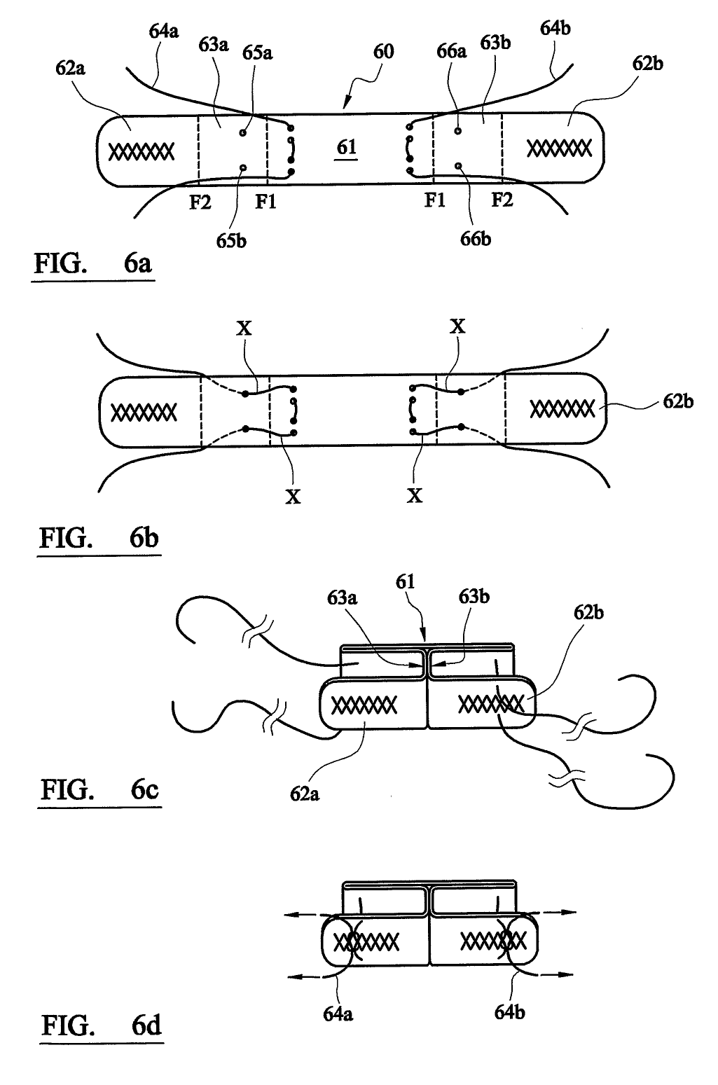

FIGS. 6a to 6d illustrate the steps involved in forming an annular repair device according to another embodiment of the invention;

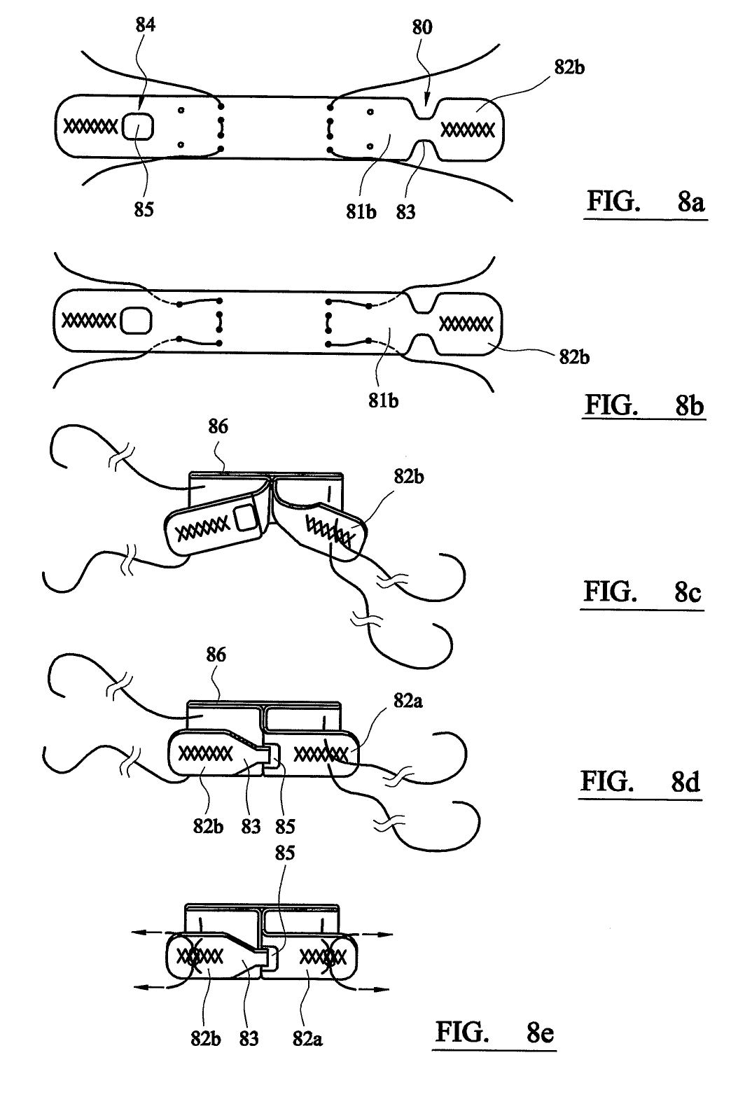

FIG. 7 illustrates the annular repair device of FIGS. 6a to 6d in-situ;

FIGS. 8a to 8e illustrate the steps involved in forming an annular repair device according to yet another embodiment of the invention;

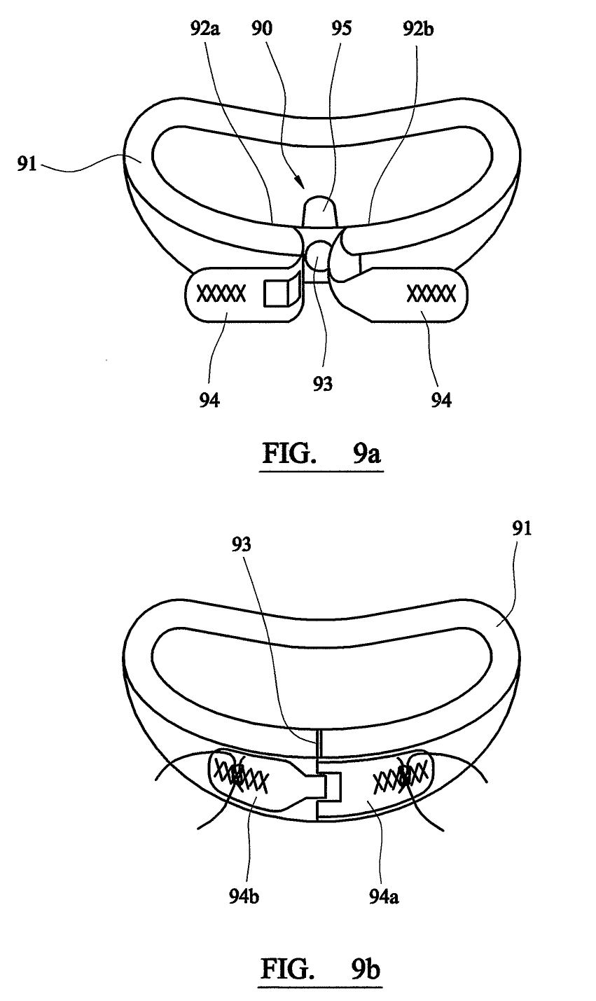

FIGS. 9a illustrates the annular repair device of FIG. 8a to 8e in-situ during deployment;

FIG. 9b illustrates the annular repair device of FIG. 9a after deployment;

FIG. 10a illustrates a further embodiment of the repair device;

FIG. 10b illustrates a variation on the embodiment of FIG. 10a; and

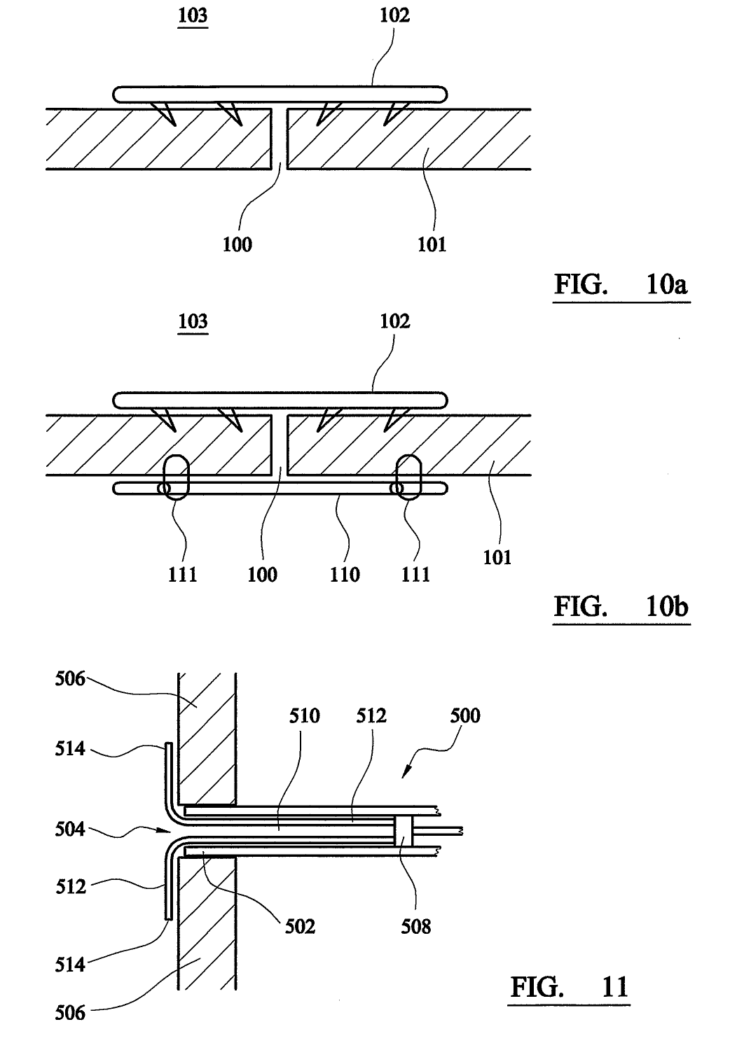

FIG. 11 illustrates an applicator for assisting in the deployment of repair devices according to the present invention;

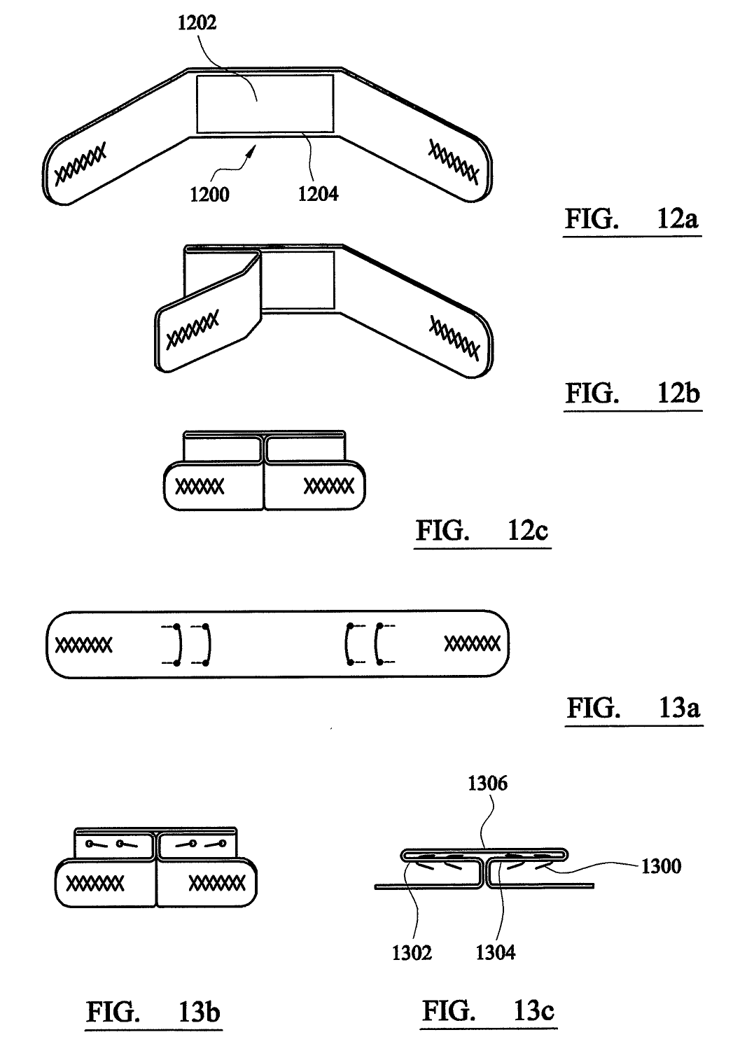

FIG. 12a to 12c illustrate an embodiment of the invention including a wire support;

FIGS. 13a to 13c illustrate an embodiment of the invention including barbs;

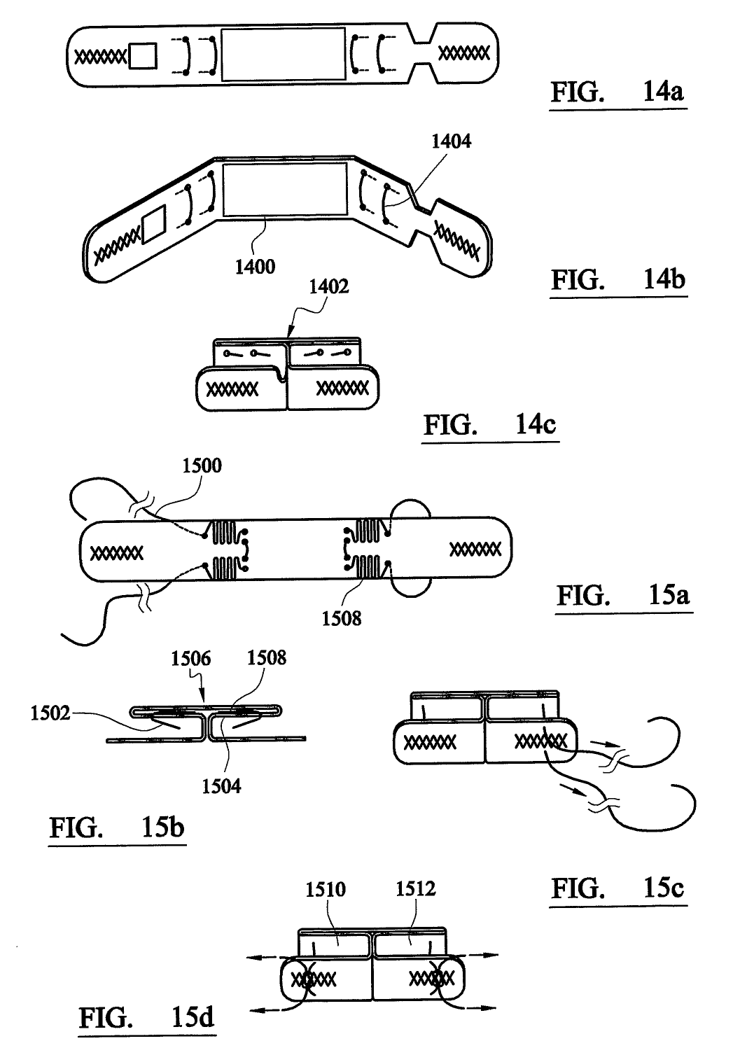

FIGS. 14a to 14c illustrate an embodiment of the invention with wire support and barbs; and

FIGS. 15a to 15d illustrate an embodiment of the invention with pre-loaded sutures.

Each of the intervertebral discs within a spine function as a spacer, as a shock absorber, and to allow motion between adjacent vertebrae. The height of the disc maintains the separation distance between the vertebral bodies. There are three functions that the intervertebral disc performs:
Proper spacing—Allows the intervertebral foramen to maintain its height, allowing the segmental nerve roots, room to exit each spinal level without compression.
Shock absorption—Not only allows the spine to compress and rebound when the spine is axially loaded (during such activities as jumping and running) but also to resist the downward pull of gravity on the head and trunk during prolonged sitting and standing.
Elasticity (of the disc) Allows motion coupling, so that the segment may flex, rotate, and laterally bend all at the same time during a particular activity. This would be impossible if each spinal segment were locked into a single axis of motion.

The intervertebral disc consists of four distinct parts. These are the nucleus pulposus, annulus fibrosus and two end plates. It should be noted that although these four sections are very much distinct in their own right the boundaries between then are not as distinct. Most investigators tend to ignore the end plates and dismiss them as merely as the barrier between the vertebrae and the parts of the disc which allow motion of the spine. However, the end plates are important in completing the structure of the disc and creating some of the boundary conditions that define the behaviour of the disc.

From around the 20th year of a persons life, the discs become completely avascular, although they show high metabolic turnover. The water content of the discs will decrease the older the person gets.

### End Plates

The end plates are composed of hyaline cartilage. This is basically a “hydrated Proteoglycan gel, reinforced by Collagen Fibrils”—Ghosh; The Biology of the Intervertebral Disc. CRC Press, ISBN 084936711523. As stated, the boundary between the annulus and end Plate is not a distinct one, under a microscope the two parts merge together, with a region which is neither one tissue nor the other.

### Annulus

The annulus is the outer ring of the disc. A strong, laminated structure of opposed layers of Collagen fibres. An annulus typically comprises around 12 laminae, with 6 provided in each direction of fibre travel. The layers are at an angle of approximately 30° on every other layer, with 30° in the opposite direction on the remaining layers. The functions the annulus performs determine the need for this type of structure. No matter which direction the vertebrae moves, there will always be some fibres in tension and some in compression. Thus, the annulus will always be acting using some fibres to stretch (they will resist stretch like an elastic band) and pull the spine back into the correct posture.

The annulus has overlapping, radial bands, not unlike the plies of a radial tyre, and this allows torsional stresses to be distributed through the annulus under normal loading, without rupture. One study suggested that the posterior part of the annulus is the weaker side, so more susceptible to damage—Tsuji; Structural variation of the annulus fibrosis. Spine 18 pp 204-210, 1993. The annulus is the strongest part of the disc.

### Nucleus

The nucleus at the centre of the disc, is a highly hydrated gel of Proteoglycans. In children and young adults, the water content can account for up to 80% of its weight—Ghosh. This gel material is a very thick fluid that is dense enough to be able to be torn. It serves the twin purposes of both direct load bearing and, by being fluid in nature, being able to change shape under loading to distribute the load to the annulus. The nucleus may only bear half the load of the FSU (functional spinal unit) with the annulus carrying the rest—Finneson; Low back pain. ISBN 0-397-50493-4, 1992. It is this shared loading that allows the disc to continue to operate even after the nucleus has been damaged.

With a damaged nucleus, the annulus has larger loads to deal with and thus degenerates faster, although the direct stresses are not sufficient to damage a healthy annulus instantly—White and Panjabi; Clinical biomechanics of the spine: Lippincott Raven, 1990. In the case of a degenerative disc, not only will the nucleus be damaged but also the annulus. In the younger patient, the annulus and nucleus are distinct separate tissues that have transition zones between them in the disc. As the patient ages the structure of each of these parts will change on a molecular level. In practice the nucleus will become more fibrous in nature and the clear distinction between the nucleus and the annulus will begin to disappear. The nucleus tissue will increasingly dry out and stiffen with advancing age. This will result in the annulus of the disc carrying a greater proportion of the compressive load in the spine than in earlier life.

### Disc Function

The disc functions by hydraulics. It is all based upon the interaction between the annulus and the nucleus. As compressive load is applied, arrow A in FIG. 1, the structure starts to crush and a pressure builds in the nucleus 1 (the intradiscal pressure). The annular fibres 3 serve a containment function to prevent the nucleus 1 from bulging or herniating. The load serves to stretch the annular fibres 3, being essentially fibres of elastic this is the way in which these fibres can resist loading.

### Degeneration of the Intervertebral Disc

Degenerative disc disease (DDD) is the process of a disc losing some of its function, due to a degenerative process, and is a very common and natural occurrence. At birth the disc is comprised of about 80% water. As ageing occurs, the water content decreases and the disc becomes less of a shock absorber, the proteins within the disc space also alter their composition. In later life, the result is often a tearing of the annulus of the disc. Typical injuries to the annulus are:

Concentric Tears These tears occur around the structure of the annulus.

Radial Tears These tears go from the nucleus through the annulus, they tend to occur in the posterior side of the disc.

Rim Lesions This type of lesion is a separation of the outer annulus from the adjacent vertebra.

The relationship between degeneration and pain is not a clear one. Theories to explain why some degenerative discs are painful include:

Injury: A tear in the annulus may release nucleus material, which is known to be inflammatory.

Nerve Ingrowth into Discs: Some people seem to have nerve endings that penetrate more deeply into the outer annulus, than others, and this is thought to make the disc susceptible to becoming a pain generator.

Loss of Height: A degenerative disc may lose height as the water content lowers. This may cause the disc to bulge outwards—pressurising the nerve roots and thus causing pain. In addition, this loss in height will have other effects that can also be pain generating:
1. The disc biomechanics will alter. Normally the nucleus pressurizes the annulus forcing the fibres into tension. However in these cases the nucleus will lose this ability and the annulus itself will be forced to carry the compressive load at that level in the spine. This will increase the stress in the annulus.
2. The load distribution through the disc will be affected by this. When the uniform distribution becomes more haphazard the load will not be carried in an even manner throughout the disc.
3. Alteration in the disc biomechanics will affect both the patient's range of motion and alter the position of the instantaneous axis of rotation in normal movements.
4. The result of these factors will usually mean increased loading on the facet joints that may in turn start to degenerate and become symptomatic.

What ever the reason behind the degeneration causing the pain, treatment to improve the position and the patient's life is important. The treatment options are discussed in more detail below.

### Herniations of the Intervertebral Disc

A herniated disc is similar to a prolapsed one, in that there is a bulge in the disc itself. However, the disc will not have collapsed in the same way. The injury is thought to be through a combination of a degenerative process and mechanical loading. The stages of disc herniation—Ibrahim; Colorado spine institute; http://www.coloradospineinstitute.com 2004; can be seen in the stages of FIG. 2. Disc degeneration, perhaps due to chemical changes associated with aging cause the disc to weaken. A bulge then forms due to this localised failure of the annulus; Stage 1. Progression of the condition can cause the nucleus to protrude out as a herniation; Stage 2. The bulge will press against the nerves in the spinal canal and cause pain that the body sees as coming from the legs. Further progression results in extrusion as the gel-like nucleus pulposus breaks through the annulus fibrosis, but remains within the disc; Stage 3. Further progression may result in the nucleus pulposus breaking through the annulus fibrosus and lying outside the disc in the spinal canal, a sequestered disc; Stage 4.

Whilst most patients with a herniation will improve without surgery in some case surgery is necessary. If surgery is required then usually the treatment will be to remove part, or all of the herniated disc, such that the nerve roots are no longer impinged.

### Surgical Treatment of Degenerative and Herniated Discs

When a disc that is showing signs of degeneration or herniation become painful a surgeon may often operate. Treatments that may be conducted include:

1. Partial discectomy—removal of local annular material to the site of a herniation.

2. Partial nucleotomy—removal of local nucleus material close to the site of the herniation.

3. Discectomy and fusion—removal of the entire disc and fusion of the disc space, used in more serious cases.

4. Other treatments such as a disc replacement or nucleus replacement—these are new treatments used as an alternative to fusion.

The surgical procedure for existing replacement artificial disc and other device insertion, requires incisions to be created in the annulus A of the intervertebral disc B. FIG. 3 illustrates typical incisions C along the top of the annulus to one side of the vertical incision D and E along the top of the annulus to the other side. Following implantation of the devices through the openings created by these incisions they are then generally sutured closed, sutures F in FIG. 3. The sutures F are applied directly to the annulus on their own. They generally act to assist retention of the device in the disc after surgery. The long term integrity of such sites and the extent to which the annulus recovers are potential issues with existing surgery and subsequent suturing of this type.

The present invention is intended as assist in the repair of the annulus in a wide variety of situations. These include repair after the following treatments:

Discectomy to repair the fissures in the annulus and prevent further disc herniation and to restore annular function and thus restore spinal biomechanics; thus preventing deformity and subsequent damage to the operated or adjacent levels;

Nucleotomy to repair fissures in the annulus and thus prevent further extrusion of nucleus material;

Artificial Disc Replacement to restore a functional annulus by inserting a device and prevent possible device migration after surgery;

Artificial Nucleus Replacement to restore a functional annulus and prevent possible device migration after insertion.

In these and other contexts the present invention aims to provide a stronger repair to the annulus than the direct suturing of the prior art. To assist in restoring the functional stiffness offered by a fully functioning annulus. To promote tissue healing in the area of the repair device, potentially through the use of polyester and the inflammatory response triggered as a result.

Referring to FIG. 4a through to FIG. 4e, a number of embodiments of one part of an annular repair device is shown in each case. The repair device is manufactured from fibres, with the fibre material being selected to actively encourage tissue in growth, for instance polyester fibres. The fibres are embroidered so as to form a strip 40.

By embroidering one or more parts of the strip 40 in different ways, different functions and properties can be provided for it. Thus in FIG. 4a, the strip 40 is reinforced 41 across most of its height and all of its length by further embroidering or denser embroidering. In the case of the FIG. 4b embodiment, the same fibres which are used to provide the embroidery are extended in four cases 42 to provide the fibres which will subsequently be used as the sutures.

In the FIG. 4c embodiment, the strip 40 has the same properties all over and the sutures 43 are in the form of separate fibres threaded through holes 44 in the strip. As shown in the FIG. 4d embodiment, specific embroidery 45 may be provided around the holes 44 to strengthen the strip 40 and protect against the sutures cutting through the strip 40 when in use. In the FIG. 4e embodiment, a reinforced panel 46 is provided along the middle of the strip 40 and serves to reinforce the position at which the sutures pass through the strip 40 and are tied in use.

The FIG. 4a to 4e embodiments only show one part of the device, strip 40. In practice this strip 40 is used together with a further strip 50, see FIG. 5a and 5b. The strips 40 and 50 are attached to one another by sutures 51, 52 in use. Suture 51 passes from front to back through first hole 53a in strip 40, then from front to back through hole 53b in strip 50, from back to front through hole 53c, from front to back through hole 53d and then from back to front through hole 53e in strip 50 before passing from back to front through hole 53f in strip 40. The two front ends of the suture 51 can then be tied of against the front of the strip 40 in the area of the reinforcement 54. A similar sequence is used in relation to suture 52 and the respective holes in the strip 40 and strip 50. Whilst the strip 50 is shown in this case as a strip of even embroidery in all places, similar embodiments to those illustrated for the front strip in FIGS. 4a to 4e or other forms could be used for the rear strip 50.

The use of the FIG. 5a device is shown in FIG. 5b. Following damage and/or surgery a weakness/incision 57 in the annulus 58 needs to be addressed. The rear strip 50 is manipulated through an incision made at the weakness 57 or an incision 57 arsing from earlier surgery (such as the insertion of a disc replacement). The rear strip 50 is positioned such that one end 50a extends to one side of the incision 57 and the other end 50b extends to the other side of the incision 57. The strip 50 maybe positioned at or within the boundary between the annulus 58 and the nucleus or device 59. The front strip 40 is positioned outside the annulus 58 and one end 40a extends to one side of the incision 57 and the other end 40b extends to the other side of the incision 57. The sutures 51 and 52 are provided attached to the rear strip 50 in the form previously described. Needles 59, which may be attached to the ends of the sutures 51, 52 or integrally provided therewith, are pushed through the part 58a, 58b of the annulus 58 between the front strip 40 and rear strip 50. The needles are passed through the strip 40 and pulled to give the desired tightness and pulling together of the front strip 40, rear strip 50 and hence annulus 58. The sutures 51, 52 are then tied off in the area of reinforcement 55.

The overall result is that the strips 40 and 50 are mechanically anchored to the annulus effectively and serve to close the incision in the annulus effectively. Firmer and more reliable positioning of the sutures is thus achieved. Furthermore, the device cannot move prior to tissue ingrowth occurring and the portion of the annulus around the incision is also kept in a constant position to assist its recovery too. The provision of the rear strip 50 across the fill width and height of the incision also means that there is a strong element present on the inside of the annulus to prevent the nucleus pressure from rupturing the repaired annulus.

An alternative embodiment of the invention is illustrated in FIGS. 6a to 6d and FIG. 7 and offers further advantages.

In this embodiment, both the front strip and rear strip are provided by the same element 60, FIG. 6a. The element 60 includes a rear strip forming portion 61, first front strip forming portion 62a and second front strip forming portion 62b. The first front strip forming portion 62a is joined to the rear strip forming portion 61 by a first link portion 63a. The second front strip forming portion 62b is joined to the rear strip forming portion 61 by a second link portion 63b.

A first suture 64a and second suture 64b pass through a set of four holes in the rear strip forming portion 61 in the same sequence as described above for FIG. 5a and 5b. From the flat form illustrated in FIG. 6a, the first part of the repair devices assembly involves passing the ends of the suture 64a through holes 65a and 65b and the ends of the suture 64b through holes 66a and 66b, FIG. 6b.

Pulling the sutures 64 and 65 shortens the lengths X, causes the rear strip forming portion 61 to fold about dotted line F1 and so form the rear strip 61. The rear strip 61 is in effect of double thickness due to the folding. The folding process brings the first link portion 63a and second link portion 63b into contact with one another, FIG. 6c. These link portions 63a and 63b thus form the double thickness link which leads to the front strip 62. The assembly up to this point may be performed outside the body.

The front strip 62 is formed by the first front strip forming portion 62a being in proximity with the second front strip forming portion 62b, FIG. 6c. The passage of the sutures 64 and 65 through the holes 66 and 67 in the front strip forming portions 62a, 62b and subsequent tightening causes the front strip forming portions 62a, 62b to be folded back, folds F2, and so form the front strip 62. The front strip 62 is very generally parallel to the rear strip 61 and very generally perpendicular to the link portions 63a, 63b. Once adjusted to the correct position, the sutures 64a, 64b can be tied off to fix the repair device. This part of the assembly is preferably performed in the body.

The assembled repair device of the FIG. 6a to 6e embodiment is shown in-situ in FIG. 7. Again the repair device has been used to address an area of weakness or to address an incision in the annulus 70. The repair device is prepared into the folded form which follows the FIG. 6b form described above. Thus the rear strip 61 has been formed from the rear strip forming portions 61a, 61b. The rear strip is then manipulated into position inside the annulus 70 and extending on both sides of the incision 71. The link portions 63a, 63b have been brought before the manipulation or are naturally brought together during the manipulation, such that they extend through the incision 71 and out of the annulus 70.

The two front strip forming portions 62a, 62b are then folded back across the front of the outside of the annulus 70. The sutures 64, 65 are then pushed through the annulus portions 73a, 73b between the rear strip forming portion 61a, 61b and front strip forming portions 62a, 62b respectively and then through the front strip forming portions 62a, 62b respectively. In this position, their tightness can be adjusted and the sutures tied off.

As well as providing the firm anchor and protection against the incision being opened by nucleus or device pressure, this embodiment has a further unexpected invention. Placing a foreign material between the two ends that are intended to join together may appear to be illogical. However, the provision of the link portions 63a, 63b extending between the cut ends of the annulus portions 73a, 73b, actually gives improved properties in the short term from the repair device and more importantly in the long term through a stronger bodily response. The layer of, for instance polyester, between the butt-jointed portions 73a, 73b of the annulus excites a strong fibrous response which should create a significantly better biological bond than would be normally be achieved by simply pulling the portions 73a, 73b together. This is particularly so given the poor blood supply to the natural annulus 70.

Another embodiment of the invention is illustrated in FIGS. 8a to 8e. This is a variation of the device set out in relation to FIG. 6a to 6d and FIG. 7 above. In this case, however, a part 80 of device toward the junction between the second link portion 81b and the second front strip forming portion 82b is provided with as a reduced width neck 83, FIG. 8a. The embroidery may be reinforced in this part 80 to account for the reduced width. The part 84 of the device toward the junction of the first link portion 81a and the first front strip forming portion 82a is provided with a through hole 85. The rim of the hole 85 may be reinforced.

The initial part of the assembly is as described above, and as illustrated in FIGS. 8b and 8c, so as to provide the rear strip 86 in the desired position inside the annulus and extending to either side of the incision.

Before the sutures are passed through any part of the front strip forming portions 82a, 82b, the front strip forming portion 82b is threaded through the hole 85, FIG. 8d. The front strip forming portion 82b is pulled through until the neck 83 comes to rest in the hole 85. The sutures are then passed through the annulus portions and the front strip forming portions 82a, 82b, tightened and tied off, FIG. 8e. The combination of the tightening and the interdigitation brings the ends of the annulus together. This embodiment provides still greater resistance to the device or nucleus causing the incision to open once more.

The device of FIGS. 8a to 8e during and after deployment is illustrated in FIG. 9a and 9b. Once this is an interdigitated device form.

The device is shown with the initial insertion of the rear strip forming portions 92a, 92b inside the nucleus space 90 complete. Before any interdigitation, the aperture 93 in the centre of the rear strip forming portions 92a, 92b needs to be closed. Before closure, this allows continued access to the nucleus space 90 inside. Once this access is no longer needed, a flap 95 is maneuvered down so as to obscure the aperture 93 from the inside. Once this step has been completed, the interdigitation is performed and the front strip forming portions 94a, 94b are then fastened to the annulus 91 using the sutures as described before.

The annular repair devices described above require relatively small incisions to be deployed. A further embodiment of the invention can be used to assist in the deployment of the repair device inside the annulus or to reduce still further the size of incision needed for deployment. In particular, an arthroscopic version of the device may be provided. In this embodiment, the rear strip forming portion is embroidered so as to surround a shape memory metal component, for instance in the form of a memory metal wire. The wire inside the embroidery or otherwise fastened thereto, has a low profile shape for insertion into the annulus. The memory metal is fixed in this state, but upon warming within the annulus assumes its other state so as to deploy the rear strip forming portion. In effect the memory metal expands to the desired profile only after insertion. The memory metal may be provided as a band around the device, as a spiral pattern, in a star shape or some other orientation. Once deployed, the device, due to the shape assumed by the memory metal would be too large to exit the implantation hole.

FIG. 10a shows a further embodiment of the invention. In this case, a fissure 100 in an annulus 101 is repaired by providing a rear strip 102 on the inside, nucleus space 103. The strip 102 is provided with a series of barbs 104. The barbs 104 are inclined so as to facilitate sliding motion into position during deployment through the fissure 100. The inclination of the barbs 104 also means that they tend to dig into the annulus 101 and anchor there to if the annulus 101 tries to move and the fissure reopen.

In the FIG. 10a form, the repair is achieved by the first strip along. It can however, be supplemented by a front strip 110 which is provided on the outside of the annulus 101 and is connected thereto by a number of sutures 111.

FIG. 11 shows one design for an applicator useful in deploying devices according to the present invention into fissures to be repaired. The applicator 500 includes a tip 502 which is hollow and can readily be inserted into a fissure 504 to be treated, the fissure being in an annulus 506. Within the applicator 500 a mount 508 is capable of sliding motion towards the tip 502 and away there from, under the control of an actuator. The repair device itself is not shown for the sake of clarity, but is held in and dispensed from the tube 510.

A pair of flexible wires or strips 512 are provided on the mount 508 and move there with. In use, the ends 514 of the wires 512 engage the limits of the rear strip of the device. As the wires 512 are advanced they carry the rear strip with them. The wires 512 are of memory metal, due to its extreme flexibility. The profile the wires 512 wish to assume is prevented by the walls of the tube 510 when they are within it. Once clear of the tip 502, however, the restraint is removed and the wires 512 can spread to their desired form. The flexibility of memory metals enables a very tight turn to be made and the flexibility of the rear strip accommodates this. The result is that the limits of the rear strip are pushed along the inside of the annulus wall. Hence the desired deployment for the rear strip is achieved.

To retract the wires 512, the direction of movement of the mount 508 is reversed and this pulls the wires 512 back into the tube 510 and leaves the rear strip in place.

In a modification, the wires 512 can abut the needles on the sutures and push them through the annulus wall from the inside once the free movement of the rear strip stops at its limit. The wires 512 could, at least in part by a component of the device rather than the applicator.

FIGS. 12a to 12c shown the makeup and folding of a modified H-shaped device. In this case the rear strip 1200 is provided on its front face 1202 with a wire support 1204. Folding of the side portions 1206 in against the rear strip 1200 forms a pocket 1208 which retains the wire in subsequent use. The wire support 1204 can be compressed or deformed to allow insertion, but returns to its rectilinear configuration in-situ to give the desired deployment. In this embodiment, the front strip is not interdigitated, but that is a possibility.

FIGS. 13a to 13c show a further embodiment which is provided with inclined barbs 1300 sandwiched between the folded across parts 1302, 1304 and the rear strip 1306. In this embodiment, the front strip is not interdigitated, but that is a possibility.

FIGS. 14a to 14c show an interdigitated version of the device with both wire support 1400 in pocket 1402 and inclined barbs 1404.

FIGS. 15a to 15d show built in sutures 1500 and needles 1502 which protrude slightly through the front 1504 of the rear strip 1506. The slack 1508 in the suture is arranged carefully and is sandwiched between the folded across parts 1510, 1512 and the rear strip 1506. In this way free running of the suture when needed is provided without the risk of knots or catches. In this embodiment, the front strip is not interdigitated, but that is a possibility.

1. A fissure repair device, the device including a first portion, a second portion and a variable link between the first portion and second portion, in which the first and second portions are portions of a common element, one of the first or second portions being formed by one or both end portions of the element, the first portion being linked to the second portion by one or more link portions, the one or more link portions being portions of the common element.

2. A device according to claim 1 in which the first portion is formed by the intermediate portion of the element and the second portion is formed by the two end portions of the element.

3. A device according to claim 1 or claim 2 in which the first portion includes a first part, second part and third part, the first portion being provided with one or more holes in the second part thereof, the first portion being provided in the first and third parts thereof with one or more further sets of holes.

4. A device according to claim 3 in which the first part and/or third part are folded against the second part, the holes in the first and third parts align with holes in the second part.

5. A device according to any preceding claim in which the second portion is provided with one or more areas of reinforcement on each of the end portions of the element.

6. A device according to any preceding claim in which a link portion is provided between the first portion and second portion.

7. A device according to claim 6 in which the link portion(s) are made of one or more materials and/or incorporate one or more materials and/or be coated with one or more materials which promote tissue in growth and/or the supply of blood.

8. A device according to claim 6 or claim 7 in which a fold is provided between a first second portion forming part and the first link portion and/or between the second link portion and the second second portion forming part, a fold is provided between the first link portion and the first portion and/or between the first portion and the second link portion, a fold is provided between a first part of the first portion and a second part of the first portion and/or between a third part of the first portion and a second part of the first portion.

9. A device according to any of claims 6 to 8 in which, when folded, the first link portion contacts the second link portion and the first and second link portions are substantially parallel to one another.

10. A device according to claim 9 in which the first and/or second link portions are at 90°+/−5° to the first portion and/or second portion.

11. A device according to any of claims 6 to 10 in which the first and/or second link portions contact the sides of the fissure.

12. A device according to any preceding claim in which the first portion and second portion define the verticals of an H shape, particularly when considered in plan view in an intervertebral disc space, and the link portion(s) define the cross bar of an H shape.

13. A device according to any preceding claim in which at least two variable links are provided, one provided between one end of the first portion and the second portion and the other is provided between the other end of the first portion and the second portion.

14. A device according to any preceding claim in which a variable link is used to vary the distance between one end of the first portion and the same end of the second portion and/or to vary the tension between one end of the first portion and the same end of the second portion and/or to pull the first portion against the inside of the annulus or a part thereof and/or to pull the second portion against the outside of the annulus.

15. A device according to any preceding claim in which a receiving space for the annulus to one side of the fissure is provided between a first end of the first portion and a first end of the second portion and a receiving space for the annulus to the other side of the fissure is provided between a second end of the first portion and a second end of the second portion.

16. A device according to any preceding claim in which the link portion or portions pass through the fissure, from inside the annulus to the outside thereof and the link portion or portions keep the sides of the fissure apart.

17. A fissure repair device, the device including a first portion, a second portion and a variable link between the first portion and second portion, in which the first and second portions are portions of a common element, the first portion being linked to the second portion by one or more link portions, the one or more link portions being portions of the common element, the second portion being formed by both end portions of the element.

18. A device according to claim 17 in which the first portion is in the forming of a first second portion forming part, first link portion, first portion, second link portion and second second portion forming part, with this being the sequence from one end to the other of the element.

19. A device according to claim 17 or claim 18 in which one of the second portion forming parts and/or the link portion connected to it, is provided with a reduced height part and/or neck part and the other of the second portion forming parts and/or the link portion connected to it, is provided with an aperture.

20. A device according to claim 19 in which the second portion forming part provided with the reduced height part and/or neck part is passed through the hole in the other second portion forming part.

21. A method of repairing a fissure in a material, the method including the steps of:
providing a fissure repair device, the device including a first portion, a second portion and a variable link;
deploying the first portion of the device inside the fissure;
deploying the second portion of the device outside the fissure;
connecting the first portion to the second portion at one or more locations using the variable link, the variable link passing through the material.
